(19) United States
(12) Patent Application Publication
(10) Pub. No.: US 2004/0035418 A1
(43) Pub. Date: Feb. 26, 2004

(54) Chemical bed design

(76) Inventor: Christofel Francois Wiid, Pretoria (ZA)

Correspondence Address: MYERS BIGEL SIBLEY & SAJOVEC, PO BOX 37428, RALEIGH, NC, 27627, US

(21) Appl. No.: 10/363,245

(22) PCT Filed: Apr. 16, 2002
(86) PCT No.: PCT/IB02/01224

(51) Int. Cl. A61M 16/00

(52) U.S. Cl. 128/202.26

(57) ABSTRACT

A reactor 10 is provided comprising a canister 12 holding a bed 14 of particulate reagent 34. The canister 12 has a peripheral wall and floor, and a bed support 16 lining the floor. The canister 12 has an open top at a level above the support 16 and at least one lower vent 11 below the support 16. An air-permeable bed divider 18 is supported on the bed support 16, the bed divider projecting upwardly from a lower extremity thereof at the bed support 16, in an upward direction.

This invention relates to the design of a chemical bed reactor, and it relates to a method of operating a chemical bed reactor. More particularly, the invention relates to such chemical reactor and method of operation, suitable for, but not limited to, employment in the context of a self-contained rescue device.

According to the invention there is provided a chemical reactor for use as, or as part of, a breathing apparatus, the reactor including a vented canister for holding a bed of particulate chemical reagent, the canister having a peripheral wall enclosing the interior of the canister and the canister having a floor for the interior of the canister;

an air-permeable bed support lining the upper surface of the floor for supporting a bed of particulate reagent in the interior of the canister, the canister having at least one upper air vent at a level above the bed support, and at least one lower air vent below the support and through the floor; and

at least one air-permeable air flow guide in the interior of the canister for guiding air flow between the bed support and the upper vent of the canister and through a bed of particulate chemical reagent supported in the interior of the canister on the bed support of the canister, each air flow guide projecting from a lower extremity thereof at or adjacent the bed support of the canister, in an upward direction.

The canister may be in the form of a rectangular open-topped box having a flat rectangular floor lined by the bed support, the box containing an air-permeable bed of particulate chemical reagent in its interior and supported on the bed support, each air flow guide having an air-permeability which is greater than the air-permeability of the bed, the open top of the box forming the upper vent of the box and the floor having a plurality of spaced evenly dispersed lower vents therethrough. Each air flow guide may be in contact with the bed support so that it rests on, and is supported by, the bed support each air flow guide projecting upwardly from the bed support to an upper extremity of the air flow guide, which upper extremity is located at a level at or adjacent the upper surface of the bed, the contact between each air flow guide and the bed support permitting air flow between the air-permeable interior of each air flow guide and the air-permeable interior of the bed support where the air flow guide rests on the bed support, the particulate material of the bed being a granular material. By granular is meant that the particulate material has particles which are more or less irregular-spherical in shape, none of the particles having a largest dimension which is more than double its smallest dimension. Each air flow guide may have a lower end portion of enlarged cross-section, the cross-section of the lower end portion tapering upwardly from a broad lower end thereof at the lower extremity of the air flow guide, to a narrow upper end thereof spaced above the broad lower end, the narrow upper end of the lower end portion merging into an upper portion of the air flow guide, which upper portion is of constant cross-section over its full height.

Each air flow guide may comprise a partition in the interior of the canister which provides a wall for separating parts of a bed of particulate chemical reagent in the interior of the canister from each other or one another. There may be a single said air flow guide which comprises a bed divider made up of a plurality of partitions in the interior of the canister which provide walls which divide the interior of the canister into a plurality of separate compartments for containing separate parts of a bed of particulate chemical reagent in the interior of the container.

The bed support and each air flow guide may be formed of air-permeable wire mesh material, being resiliently flexible. The bed support and each air flow guide may be formed of a plurality of resiliently flexible layers of knitted wire mesh material. Two adjacent layers of the knitted wire mesh of each air flow guide may be spaced from each other by an air space.

The reactor may be for use as, or may be part of, a self-contained rescue device of the re-breathing type, for use with a particulate chemical reagent having air-purification properties.

Further according to the invention, in a chemical reactor for use as, or as part of, a breathing apparatus, the reactor including a vented canister for holding, in the interior of the canister on an air-permeable bed support lining the upper surface of a floor of the canister, a bed of particulate chemical reagent, which bed is air-permeable and whose particles are capable of deteriorating into a condition wherein the bed has reduced air-permeability, there is provided the method of operation which comprises guiding air through the interior of the bed along air-permeable interiors of one or more air-permeable air flow guides, each of which air flow guides is located in the bed and each of which air flow guides extends between two positions in the bed which are respectively at or adjacent the bed support and at or adjacent the upper surface of the bed.

The guiding of the air may include guiding it along air flow passages in the interiors of the air flow guides. The method may include holding the bed in one or more compartments in the interior of the canister, one or more of the compartments having at least one flexible wall for absorbing shocks and/or absorbing vibrations, for reducing damage to, and consequent size reduction and deterioration of, the particles of the bed arising from said shock and/or vibration.

The method may include promoting said shock—and/or vibration absorption by holding a plurality of parts of the bed in a plurality of compartments, each of which has at least one resiliently flexible wall supporting the particles of the bed and dividing the bed into said parts. Each flexible wall may be air-permeable, the guiding of the air being along the air-permeable interiors of the flexible walls.

The invention is now described, by way of an example, with reference to the accompanying diagrammatic drawings, in which:

FIG. 1 shows schematically a sectional side elevation of a chemical reactor in accordance with the invention;

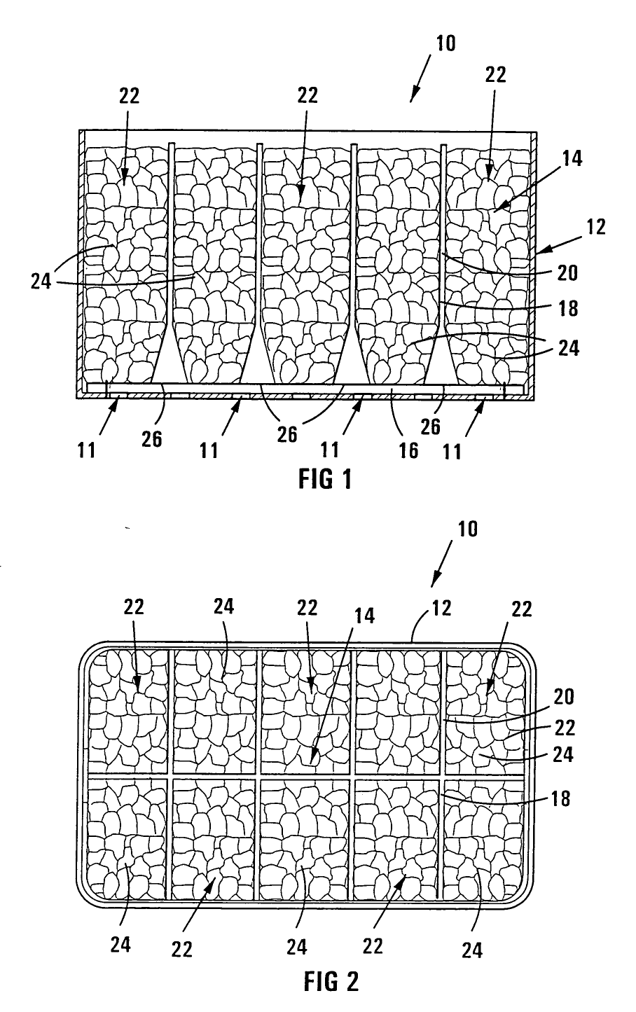

FIG. 2 shows schematically a plan view of the reactor of FIG. 1;

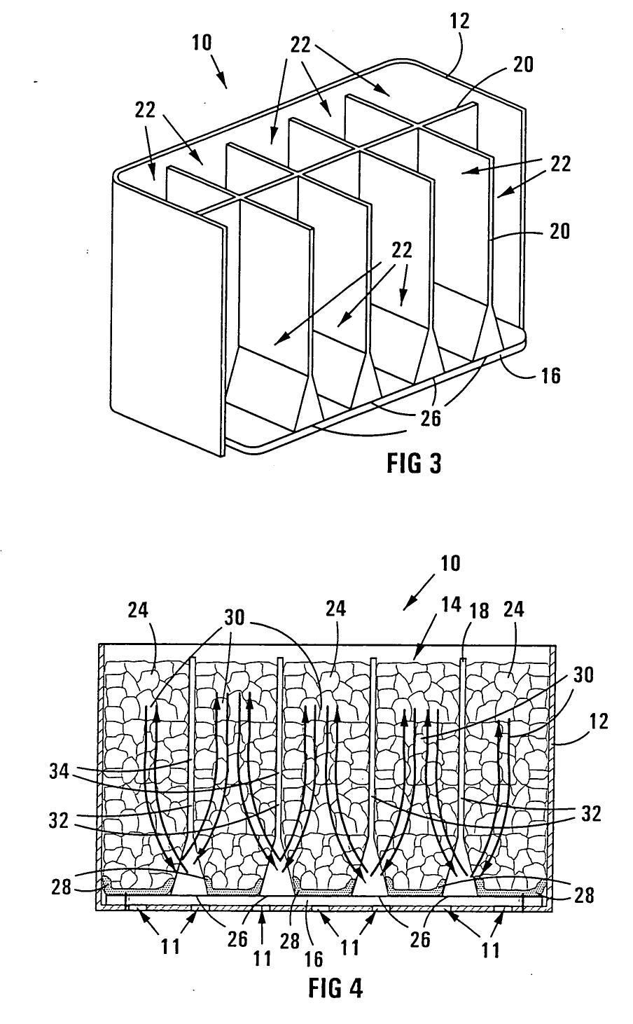

FIG. 3 shows schematically a three-dimensional view of the reactor of FIGS. 1 and 2, without reactant; and

FIG. 4 shows schematically a sectional side view of the reactor of FIGS. 1-3 in use.

Referring first to FIGS. 1-3, a chemical reactor in accordance with the invention, is generally indicated by reference numeral 10. The reactor 10 has a housing in the form of an open-topped rectangular box having a peripheral wall made up of a pair of longer side walls and a pair of shorter end walls, the box being elongated. The box has an elongated rectangular floor provided with a plurality of evenly dispersed spaced vents in the form of circular openings 11 therethrough.

The reactor 10, is for use in a self-contained rescue device of the re-breathing type comprising a rescue pack (not shown). The housing or box is designated 12 and houses a chemical reagent bed 14. The reagent bed 14 rests on an air-permeable bed support 16 that lines the floor of the box 10 and is substantially flat. A bed divider 18 forming an air-flow guide has air-permeable walls 20 which are substantially perpendicular to, and rest on, the bed support 16. The walls 20 provide a plurality of partitions which define ten compartments 22 in the box 12. A granular chemical reagent 24, forming the bed 14, is contained in each compartment 22, the bed 14 being air-permeable such that air can flow through the bed 14 of reagent 24, through the interiors of the walls 20 of the divider 18, and through the support 16 at contact regions 26 between the walls 20 of the divider 18 and the support 16. The support 16 and the divider 18 are each made of a plurality of layers (not shown) of knitted wire mesh and the divider walls 20 have enlarged lower end portions adjacent the support 16, the end portions tapering in a cross-reference section upwardly from the regions 26 to narrow upper end portions of the walls 20, the upper end portions being of constant cross-section.

Referring also to FIG. 4, a reactor used in a self-contained rescue pack is generally indicated by reference numeral 10, the same reference numerals designating the same parts as in FIGS. 1-3, unless otherwise specified.

In use, air will flow up and down through the bed 14, between the vents 11 in the floor of the box 12 and the open top of the box 12, passing through the air-permeable bed 14 of reagent 12, and through the air-permeable support 16 and air-permeable walls 20 of the divider 18. The granular reagent 24 of the reactor 10 in use deteriorates at least partially into a fluid paste-like state and flows downwardly towards the bottom of each compartment 22, where it is shown at 28, thereby restricting air flow through the bottom of each compartment 22 and through the support 16. Sufficient air flow, indicated by the arrows 30, is maintained through the bed 14 of granular chemical reagent 24 by providing air flow passages at 32 in the bed 14 of reagent 24. The air flow passages at 32 are provided by empty spaces at 34 between knitted layers of the wire mesh walls 20 dividing the bed 14, and between the knitted layers of the wire mesh bed support 16 supporting the bed 14.

The wire mesh forming the divider 18 is resiliently flexible. Thus, size reduction and the formation of powder from the granular chemical reagent 24 arising from shock and vibration are inhibited by containing the granular reagent 24 within the compartments 22 defined by the resiliently flexible divider 18 which absorbs shocks and vibrations.

Powder formation and associated deterioration of the chemical reagent 24 is further inhibited by filling the compartments 22 defined by the partitions 20 of the divider 18 with granular reagent 24 so as to leave the partitions 20 and bed support 16 in a resiliently stressed state. The wire mesh partitions 20 and bed support 16 are resiliently flexible and remain resiliently stressed, so that they are urged continuously to contact with the reagent 24 of the bed 14, despite any decrease in its volume arising from deterioration of the reagent 24.

The main advantages of this invention promise to be that sufficient air flow through the bed 14 of granular chemical reagent 24 is permitted, despite deterioration of the reagent 24, and that contact between the air and the reagent 24 can at least partly be maintained as a result of air flow through the layers of knitted wire mesh of the support 16 and divider 18, where the support 16 and divider 18 are in contact with the material 24. A further advantage of the invention is that mechanical deterioration of the granular reagent 24 is inhibited when the reactor 10 is subjected to shocks and/or vibrations. This inhibition is by virtue of the shock-absorbency of the support 16 and divider 18. The knitted wire mesh of the support 16 and divider 18 also allow powder and dust from the bed 14 to fall away, out of the reactor 10 through the openings 11, while containing larger granules inside the box 12 of the reactor 10.

1. A chemical reactor for use as, or as part of, a breathing apparatus, the reactor including a vented canister for holding a bed of particulate chemical reagent, the canister having a peripheral wall enclosing the interior of the canister and the canister having a floor for the interior of the canister;
an air-permeable bed support lining the upper surface of the floor for supporting a bed of particulate reagent in the interior of the canister, the canister having at least one upper air vent at a level above the bed support, and at least one lower air vent below the support and through the floor; and
at least one air-permeable air flow guide in the interior of the canister for guiding air flow between the bed support and the upper vent of the canister and through a bed of particulate chemical reagent supported in the interior of the canister on the bed support of the canister, each air flow guide projecting from a lower extremity thereof at or adjacent the bed support of the canister, in an upward direction.

2. A reactor as claimed in claim 1, in which the canister is in the form of a rectangular open-topped box having a flat rectangular floor lined by the bed support, the box containing an air-permeable bed of particulate chemical reagent in its interior and supported on the bed support, each air flow guide having an air-permeability which is greater than the air-permeability of the bed, the open top of the box forming the upper vent of the box and the floor having a plurality of spaced evenly dispersed lower vents therethrough.

3. A reactor as claimed in claim 2, in which each air flow guide is in contact with the bed support so that it rests on, and is supported by, the bed support each air flow guide projecting upwardly from the bed support to an upper extremity of the air flow guide, which upper extremity is located at a level at or adjacent the upper surface of the bed, the contact between each air flow guide and the bed support permitting air flow between the air-permeable interior of each air flow guide and the air-permeable interior of the bed support where the air flow guide rests on the bed support, the particulate material of the bed being a granular material.

4. A reactor as claimed in any one of the preceding claims, in which each air flow guide has a lower end portion of enlarged cross-section, the cross-section of the lower end portion tapering upwardly from a broad lower end thereof at the lower extremity of the air flow guide, to a narrow upper and thereof spaced above the broad lower end, the narrow upper end of the lower end portion merging into an upper portion of the air flow guide, which upper portion is of constant cross-section over its full height.

5. A reactor as claimed in any one of the preceding claims, in which each air flow guide comprises a partition in the interior of the canister which provides a wall for separating parts of a bed of particulate chemical reagent in the interior of the canister from each other or one another.

6. A reactor is claimed in claim 5, in which there is a single said air flow guide which comprises a bed made up of a plurality of partitions in the interior of the canister which provide walls which divide the interior of the canister into a plurality of separate compartments for containing separate parts of a bed of particulate chemical reagent in the interior of the container.

7. A reactor as claimed in any one of the preceding claims, in which the bed support and each air flow guide are formed of air-permeable wire mesh material and are resiliently flexible.

8. A reactor as claimed in claim 7, in which the bed support and each air flow guide are formed of a plurality of resiliently flexible layers of knitted wire mesh material.

9. A reactor as claimed in claim 8, in which two adjacent layers of the knitted wire mesh of each air flow guide are spaced from each other by an air space.

10. A reactor as claimed in any one of the preceding claims, which is part of a self-contained rescue device of the re-breathing type, for use with a particulate chemical reagent having air-purification properties.

11. In a chemical reactor for use as, or as part of, a breathing apparatus, the reactor including a vented canister for holding, in the interior of the canister on an air-permeable bed support lining the upper surface of a floor of the canister, a bed of particulate chemical reagent, which bed is air-permeable and whose particles are capable of deteriorating into a condition wherein the bed has reduced air-permeability, the method of operation which comprises guiding air through the interior of the bed along air-permeable interiors of one or more air-permeable air flow guides, each of which air flow guides is located in the bed and each of which air flow guides extends between two positions in the bed which are respectively at or adjacent the bed support and at or adjacent the upper surface of the bed.

12. A method as claimed in claim 11, in which the guiding of the air includes guiding it along air flow passages in the interiors of the air flow guides.

13. A method as claimed in claim 11 or 12, which includes holding the bed in one or more compartments in the interior of the canister, one or more of the compartments having at least one flexible wall for absorbing shocks and/or absorbing vibrations, for reducing damage to, and consequent size reduction and deterioration of, the particles of the bed arising from said shock and/or vibration.

14. A method as claimed in claim 13, which includes promoting said shock and/or vibration absorption by holding a plurality of parts of the bed in a plurality of compartments, each of which has at least one resiliently flexible wall supporting the particles of the bed and dividing the bed into said parts.

15. A method as claimed in claim 13 and claim 14, in which each flexible wall is air-permeable, the guiding of the air being along the air-permeable interiors of the flexible walls.

16. A reactor as claimed in claim 1, substantially as described and as illustrated herein.

17. A method is claimed in claim 11, substantially as described and as illustrated herein.
